(19) United States
(12) Patent Application Publication
(10) Pub. No.: US 2005/0075642 A1
(43) Pub. Date: Apr. 7, 2005

(54) Bone smoothing method and system

(76) Inventors: Jeffrey C. Felt, Greenwood, MN (US); Mark A. Rydell, Turnpike Road, MN (US); David Griffin, Vero Beach, FL (US)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(21) Appl. No.: 10/499,137

(22) PCT Filed: Dec. 19, 2002
(86) PCT No.: PCT/US02/40883

Related U.S. Application Data
(60) Provisional application No. 60/341,070, filed on Dec. 19, 2001.

(51) Int. Cl. A61B 17/58

(52) U.S. Cl. 606/89

(57) ABSTRACT

A method and system for preparing a bone surface, as by smoothing the surface, in preparation for contacting and/or receiving a prosthetic implant. The smoothing system and method are particularly well suited for use with articulating joints, in which a polymeric biomaterial such as polyurethane is implanted and retained upon one (supporting) bone surface, in order to provide a corresponding surface for opposing, articulating bone. Smoothing condylar bone, for instance, can significantly decrease the friction between a the condylar surface, and particularly one that is itself diseased or damaged, and to remove osteophytes and entheseophytes.

### CROSS REFERENCE TO RELATED APPLICATIONS

The present application is a continuation in part of U.S. Provisional Application Ser. No. 60/341,070, filed Dec. 19, 2001, and a continuation in part of U.S. application Ser. No. 10/167,963 filed Jun. 12, 2002, the disclosures of both of which are incorporated herein in their entireties.

### TECHNICAL FIELD

In one aspect, this invention relates to biomaterials formed either in vivo, as by the delivery of a curable biomaterial, or formed ex vivo for implantation and use within the body. In another aspect, this invention further relates to the field of orthopedic implants and prostheses, and more particularly, for implantable polymeric materials for use in articulating orthopedic joints. In yet another aspect, the invention relates to methods and systems for preparing a joint site, including for smoothing or texturing a bone surface to be placed in contact with an implantable prosthetic material.

### BACKGROUND OF THE INVENTION

Applicant has previously described, inter alia, prosthetic implants formed of biomaterials that can either be delivered and finally cured in situ, or that can be prepared (e.g., partially or fully cured) outside the body, and implanted for final use. Such biomaterials have particular use in the repair of the tibial surface of the knee, and include the use of polyurethane biomaterials. See for instance, U.S. Pat. Nos. 5,556,429, 5,795,353, 5,888,220, 6,079,868, 6,140,452, 6,224,630 and 6,248,131 as well as published International Application Nos. WO 95/30388 and WO 97/26847 and International Application PCT/US97/20874 filed Nov. 14, 1997, and U.S. application Ser. No. US 2002/0173852A1 published Nov. 21, 2002). See also, Applicant's “Porous Biomaterial and Biopolymer Resurfacing System” (PCT/US99/10004), as well as “Implantable Tissue Repair Device (PCT/US99/11740), and “Static Mixer” (PCT/US99/04407) applications.

Separately, U.S. Pat. No. 6,206,927 describes a self-centering meniscal prosthesis device suitable for minimally invasive, surgical implantation into the cavity between a femoral-condyle and the corresponding tibial plateau is composed of a hard, high modulus material shaped such that the contour of the device and the natural articulation of the knee exerts a restoring force on the free-floating device. In what Appears to be a related manner, Sulzer has introduced a unicompartmental interpositional spacer to treat osteoarthritis in the knee. See “Little Device Could Pack a Big Punch”, Sulzer Medica Journal Edition February 2000.

Over recent years, a variety of devices and systems have been developed and introduced for use in minimally invasive surgery, including orthopedic surgery. An array of orthopedic instruments are manufactured by companies such as MicroAire, Stryker, Zimmer/Hall, Aesculap, Codman, 3M, and Dyonics.

Generally, such cutting and shaping devices are used in open surgical procedures, e.g., for the purpose of resecting bone in order to provide partial or total knee replacements. See, for instance, Spotomo, et al., U.S. Pat. No. 6,319,256, which describes a bone rasp for a femur head prosthesis. See also, Braslow, et al., U.S. Pat. No. 6,059,831, which describes a method of implanting a uni-condylar knee prosthesis, including the steps of preparing the bone surfaces of both the femoral and tibal compartments. The femoral compartment is prepared by making a distal cut, a posterior cut, and a posterior chamfer cut. The tibial compartment is prepared by using a cutting guide and following the sclerotic bone formation on the proximal tibia. See also, Engh, et al., which describes an apparatus and method for “sculpting” the surface of a joint.

Surgical orthopedic instruments can also include arthroscopic and other minimally invasive instruments such as reciprocating bone saws, rasps, and the like. For instance, Shechter et al. (U.S. Pat. No. 5,685,840) describes a method and apparatus for minimally invasive tissue removal that includes motor driven reciprocating cutting blade, having the ability to control the frequency of reciprocation using an integrated feedback control system, and including optional rasp or tissue morcelator cutting heads.

Surgical, including minimally invasive, devices have also been describe to achieve bone cutting or smoothing using non-mechanical means, as by the use of lasers for instance. See, for instance, “Parameters for Safe Application of the 2.1 μm Holmium:YAG Laser for Chondroplasty of the Medial Femoral Condyle”, Janecki et al., Arthroplasty Arthroscopic Surgery 9(1):1-6, 1-998.

Applicant's own applications, including for instance the above-captioned published US application, provide a variety of options regarding the preparation of the joint site, prior to the delivery of a curable biomaterial or a cured (or partially cured) prosthetic implant. These options include, for instance, the preparation of the tibial surface for receipt and retention of a polymeric implant, and preparation of the corresponding condylar surface, which will articulate with the newly provided prosthetic implant surface, in order to remove diseased or damaged condylar tissue. Applicant's application provides, for instance, that “the partially or fully cured preformed component(s) and/or curable biomaterial and related molds, the method and system of this invention include the optional use of various additional materials and/or steps, e.g., to prepare the bone surface itself, to provide suitable interfaces (e.g., adhesive interfaces and/or protrusions that can be further secured to the joint site or by smoothing of the femoral condyle or tibial plateau as needed), to treat one or more surfaces in order to provide them with different or improved properties as compared to the inherent properties of the material providing the surface, and the like. Examples of such materials include, for instance, the use of adhesive materials, tissue in-growth stimulators, and fibrous materials (e.g., webs adapted to tether the implant and/or to facilitate fibrous tissue ingrowth).” While such repair and resurfacing methods and materials provide useful benefits, there remains the need to minimize the degradation that can occur with such implants, and particular polymeric implants, by the long term contact of the newly formed surface with the corresponding surface of an articulating bone. Applicant has found, for instance, that imperfections, and particularly osteophytes, upon the condylar surface of the knee can tend to degrade a corresponding tibial implant over time, releasing both polymeric and tissue debris. What is clearly needed is a method of preparing the joint site, in a manner that is easy to perform, and that is both technically and cost effective.

### BRIEF DESCRIPTION OF THE DRAWING

In the Drawing:

FIG. 1 shows top (a), side (b), bottom (c) and end (d) perspectives of a preferred smoothing device-of this invention.

FIG. 2 shows a detailed perspective of the distal, smoothing portion of the bottom perspective of FIG. 1, having a fine smoothing texture.

FIG. 3 shows an detailed perspective of an optional distal, smoothing portion of the bottom of FIG. 1, having instead a coarse smoothing texture.

FIGS. 4-6 correspond with FIGS. 1, 12 and 13, respectively from Applicants above-captioned U.S. application Ser. No. 10/167,963.

FIG. 7 shows an alternative embodiment in which movement of the knee itself is used to achieve smoothing using a device as described herein.

FIG. 8 shows an alternative embodiment in which the tibial surface is smoothed, and shaped, using a device as described herein.

### SUMMARY OF THE INVENTION

The present invention provides a method and system for preparing a bone surface, as by smoothing the surface, in preparation for contacting and/or receiving a prosthetic implant. The method and system have particular use in combination with Applicant's own method and system for repairing and resurfacing orthopedic joints, for instance, as described in published U.S. application Ser. No. US 2002/0173852A1 (published Nov. 21, 2002), the entire disclosure of which is incorporated herein by reference.

Both the smoothing and repair/resurfacing systems and methods are particularly well suited for use with articulating joints, in which a polymeric biomaterial such as polyurethane material is implanted and retained upon one (supporting) bone surface, in order to provide a corresponding surface for opposing, articulating bone. The ability to smooth the surface of either supporting (e.g., tibial) bone and/or articulating (e.g., condylar) bone, provides a variety of benefits. Smoothing condylar bone, for instance, can significantly decrease the friction between an implant and the condylar surface, and particularly one that is itself diseased or damaged, and to remove osteophytes and entheseophytes. The device can be used to remove or minimize ridges and pressure points, and hence the bone will be less stressful on the implant over the course of its long term use.

In a particularly preferred embodiment, the invention provides a smoothing device for preparing one or more surfaces within an articulating joint site, the device comprising a substantially flat, straight or curved, blade having a proximal portion adapted to be hand held and/or attached to a powered surgical instrument, and a distal portion having at least one major surface provided with a texture adapted to smooth cartilage within the joint site. The device can be adapted (e.g., have dimensions and other properties suitable) for use with one or more surfaces of the bones in the knee joint, and is particularly adapted for use in smoothing the condylar surface.

The device can be fabricated from any suitable material, or combination of materials, for instance, from metals such as surgical stainless steel and/or from polymeric materials. The distal portion can be textured in any suitable manner, e.g., by forming or attaching an abrasive material, by providing a plurality of closely spaced holes extending through the width of the blade, or by forming (e.g., machining or etching) a plurality of closely spaced ridges or pegs. The device can be provided with any suitable combination of strength and rigidity, for instance, permitting it to be shaped (e.g., curved) either at the time of manufacture, and/or by the surgeon, either prior to positioning the device within the site (as by bending) and/or within the site itself (as by fingertip pressure).

The device is preferably also adapted for use in a powered instrument, such as a reciprocating, oscillating or sagital saw instrument. In a preferred device, the proximal portion is provided in the form of generally circular shaft, adapted to be fixably and releasably positioned within a powered surgical instrument.

In a related aspect, the present invention further provides a method of smoothing bone, comprising providing a device as described herein, and using the device to smooth at least one surface of an articulating joint, prior to providing a surface implant upon an opposing bone surface. In yet another aspect, the invention provides a kit comprising one or more devices as described herein, e.g., devices having different dimensions and/or textures, and further comprising one or more polymeric implants for fixation to the surface of opposing bone, in a manner that contacts the smoothed bone over the course of the implant's use.

### DETAILED DESCRIPTION

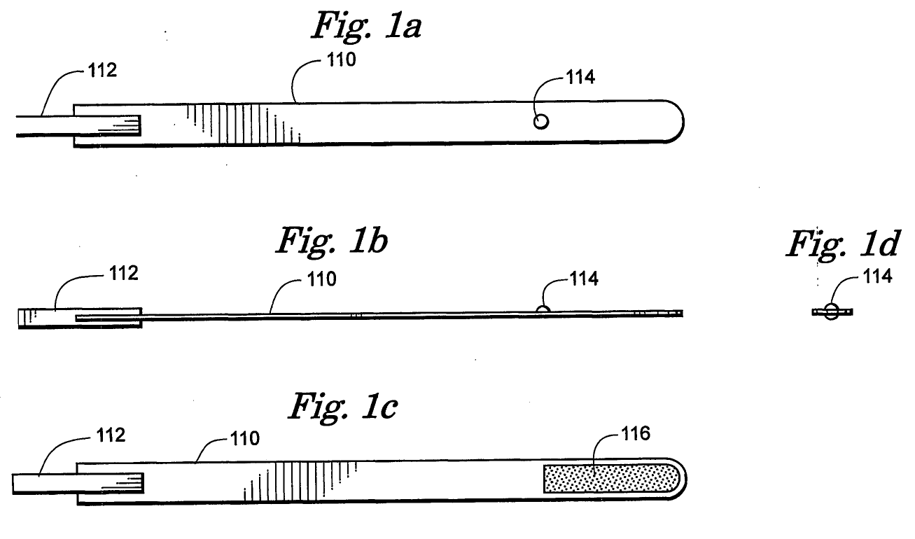

A smoothing device of the present invention will be further described with reference to the Drawing, where FIG. 1 shows top (a), side (b), bottom (c) and end (d) perspectives -of a-preferred smoothing device of this invention. As shown, the device is comprised of a substantially flat metallic (e.g., stainless steel) blade-shaped piece (110), having on its proximal end an integral or fixably attached shaft (112) for attachment to a powered surgical instrument, such as a reciprocating saw device (not shown), and on its distal end both a center punched hole or dimple (114) denoting the proximal end of the smoothing portion, and on one major surface the smoothing portion itself (116).

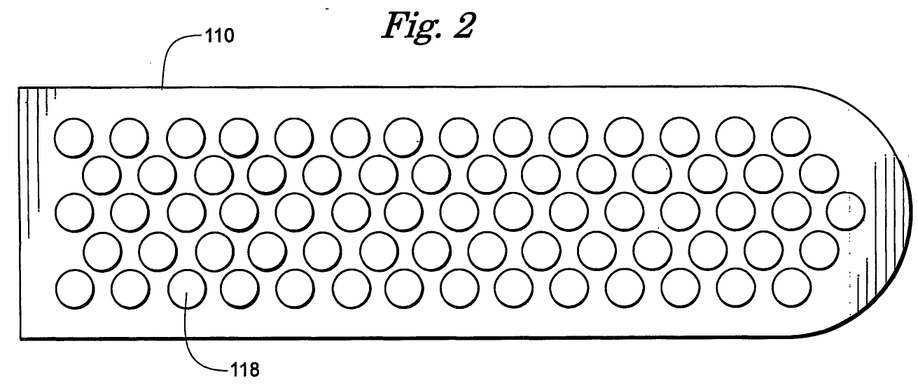

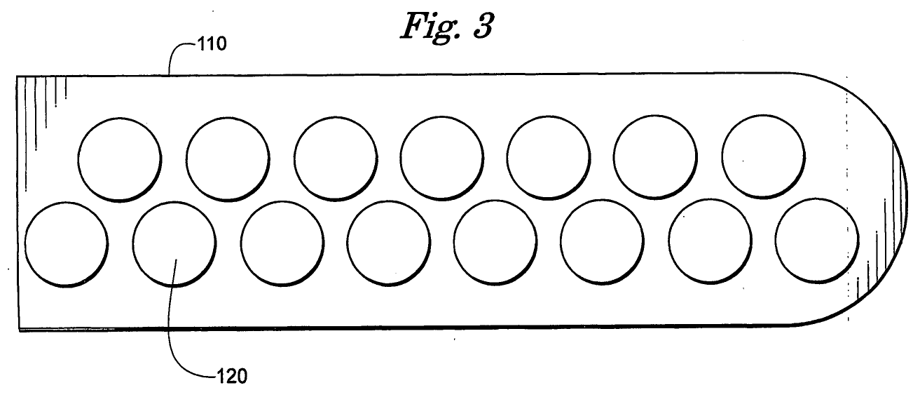

FIGS. 2 and 3 show detailed perspectives of the distal, smoothing portion of the bottom perspective of FIG. 1, having both fine and course smoothing textures, respectively.

The device can have any suitable dimensions, e.g., with a blade length of between about 100 mm and 150 mm (117 mm as shown), including a grit portion of between about 20 mm and about 40 mm in length (27 mm as shown). The blade can be of any suitable width, e.g., between about 5 mm and about 10 mm (7.5 mm as shown), and have a thickness (proximal to the smoothing portion) sufficient to provide desired flexibility, e.g., between about 0.3 mm and about 5 mm (0.56 mm as shown).

The smoothing portion of FIG. 2 is shown as a plurality of closely placed pegs (here shown having slightly raised edges) extending through the blade, machined to have a total thickness of between about 0.5 and about 1 mm (0.77 as shown), having staggered centers and each having a diameter of 0.8 mm with a height of 0.28 mm. A smoothing portion having such dimensions can provide a relative fine smoothing portion, as compared to the coarser portion shown in FIG. 3, where the pegs again have staggered centers, with centers separated by 1.625 mm along the x-axis and by 2.5 mm along the y-axis, as well as each having a diameter of 2.2 mm, a height of 0.28 mm.

Osteophytes and entheseophytes are proliferative cartilaginous and bony growths formed by acute injury, chronic irritation or degeneration of joint tissue. They can destabilize a joint, by undergoing endochondral ossification, and have the potential to cause substantial damage by encroaching into the joint space or breaking off and becoming loose bodies. When present in contact with an implant as described herein, and particularly in articulating contact over long periods of time, these structures can result in accelerated degradation of the implant surface.

A system of this invention includes a reciprocating blade device having one or more major surfaces, at least one of the major surfaces being provided in a textured form suitable to compress and/or remove tissue, including optionally bone, in order to smooth the bone surface. The device itself can be formed of any suitable material, and is preferably formed of metal or other suitably flexible but strong material.

The abrasive texture can be formed in different ways, either within and/or upon the material forming the reciprocating blade itself, or as a separate layer or portion attached thereto. The textured surface can, for instance, include a series of machined holes through a substantially flat metallic blade, or as a plurality of raised pegs, rising above but preferably integral with the plane of the device itself. By varying the size and spacing of the holes different cutting effects can be achieved. The holes can be made using various techniques including laser, electrical discharge machining (EDM), or chemical etching. Creating a plurality of raised portions with sharp edges can also be used to provide suitable texture. Similarly, conventional processes of chemical etching and die stamping can be used to make this type of abrasive surface.

Preferably, the smoothing portion is textured to a desired extent, sufficient to permit it to be used for smoothing bone in reciprocating movement, e.g., reciprocating at high speed and with an excursion of between about ½ mm and about 10 mm, and more preferably between about 1 mm and about 5 mm. Ideally, the surface is itself of a type that will not dislodge any particles (e.g., abrasive debris) in the course of its use. In turn, the device is preferably adapted to be used in a minimally invasive fashion, and without the substantial release of debris from the device surface.

The blade device can be adapted to be attached to and operated by a conventional reciprocating cutting instrument, such as those available from Medtronic, Inc. as the “Midas Rex” line of powered surgical instrumentation, or from Stryker as the Model 2106 reciprocating saw.

A reciprocating smoothing system of this invention has particular use in the preparation of a joint site for receiving a polymeric implant of the type described in Applicant's co-pending U.S. application Ser. No. 10/167,963 filed Jun. 12, 2002. The system of this invention can be used for smoothing the surface of a bone in the knee,hip, spine, wrist, elbow, shoulder or ankle.

A smoothing system of this invention can be used with an array of methods and systems for the creation or modification of the wear surface of orthopedic joints, -including one or both of two articulating surfaces and/or portions thereof, and particularly articulating joints such as the knee. In one preferred embodiment, the repair/resurfacing method relies, at least in part, upon the manner in which the various stages of curing a curable biomaterial, and in turn, the various stages of forming a component from the cured or curing biomaterial, can be correlated and optimized in a desired manner. In turn, such a method provides the ability to both generally and specifically form the component for use in situ.

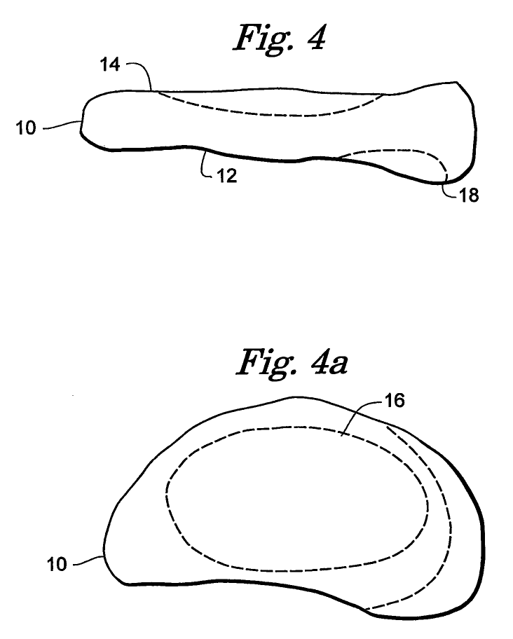

FIG. 1 of Applicant's above-captioned, co-pending published US application (reproduced as FIG. 4 herein) shows a top and side perspective of a preferred preformed knee implant (10) prepared using an ex vivo mold according to the present invention. The implant provides a first major surface (12) adapted to be positioned upon the tibial surface, and a generally planar second major surface (14) adapted to be positioned against the femoral condyle. In a typical embodiment, the second major surface, in turn, is preferably provided with a femoral glide path (16) to facilitate its performance in situ, in the form of a generally central (e.g., oval) depression about 0.5 mm, or more preferably about 1 mm to about 5 mm deep at its lowest point (2 mm as shown) and about 20 mm, and more preferably about 30 mm to about 50 mm in length by 10 mm to 30 mm in width (40 mm by 20 mm as shown). Those skilled in the art, given the present description, will readily determine the actual dimensions for optimal use, in both absolute and relative terms, depending on such factors as the actual joint size and desired results (e.g., angular correction). As shown, the implant is also provided with a tibial projection (18), adapted to catch the posterior portion of the tibial plateau by extending over the rim of the tibial plateau distally. The body of the implant can have dimensions on the order of between about 35 mm, and preferably about 40 mm to about 60 mm in the anterior-posterior dimension, between about 20 mm, and preferably 30 mm to about 35 mm, or even about 40 mm in the medial-lateral dimension, and a maximum thickness (at the posterior lip of between about 8 mm, more preferably about 10 mm, and about 20 mm, or about 2 mm to about 4 mm (e.g., 3 mm) greater than the thickness of the implant at the center. As a result, it can be seen that fixation is accomplished by effectively capping the tibial plateau with one or more projections extending distally over the rim of the plateau.

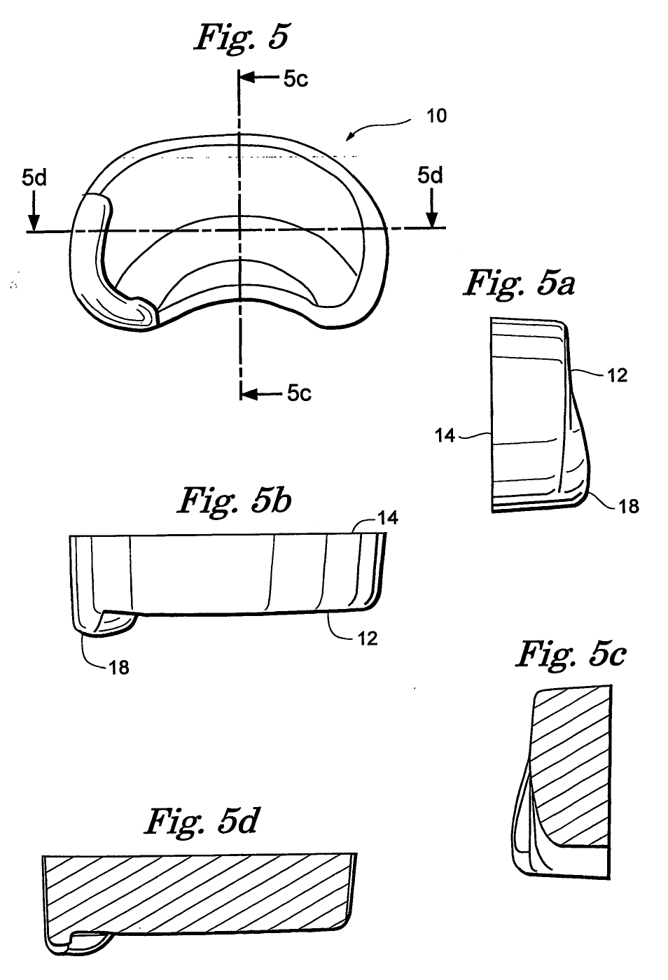

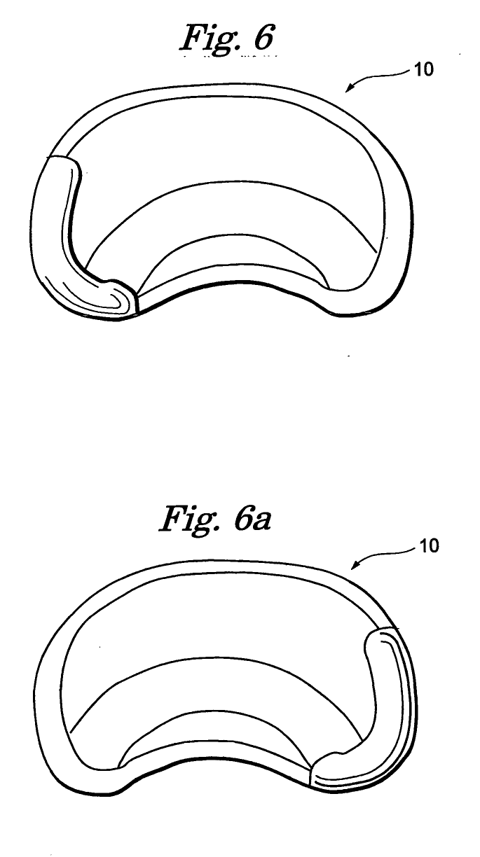

FIG. 12 of Applicant's co-pending published application (reproduced as FIG. 5 herein) shows various views of a particularly preferred implant of the present invention, of the general type shown in FIG. 1 and described above, including a top plan view (a), front plan view (b), side plan view (c), section view (d) taken along A-A of FIG. 12(a) and a section view (e) taken along C-C of FIG. 12(a). FIG. 13, in turn, (reproduced as FIG. 6 herein) show side by side top plan views (a) and (b) of corresponding implants for the left and right knees, respectively. Reference numbers for the various portions correspond to those described in FIG. 1, including preformed knee implant (10), the first major surface (12) adapted to be positioned upon the tibial surface, and a generally planar second major surface (14) adapted to be positioned against the femoral condyle. The second major surface is shown having a femoral glide path surface (16) to facilitate its performance in situ, adapted to form a generally central depression having the dimensions described herein. The glide path is fully formed in situ, by a suitable combination of both shaping and repositioning of the femoral condyle in the manner described herein.

An implant of the type shown provides various benefits, including the correction of varus deformities, based in significant part upon the presence and configuration of the posterior mesial lip (18), and the cutout (kidney bean shaped) for the intercondylar eminence. The tibial projection (18) is adapted to catch the posterior portion of the tibial plateau. The implant itself has dimensions as provided herein, and can be provided using one of a collection of molds of multiple sizes and/or styles in accordance with the various parameters of the present invention. A kit can be provided containing molds of various sizes, e.g., varying by 1 mm or 2 mm 30 increments in thickness and providing a range of anterior to posterior dimensions.

Such molds can also be used to provide implants having bottoms of various shapes, e.g., either a flat or curved bottom, and for either the left or right knee.

An implant such as the configuration shown in FIG. 5 herein is preferably used in a method that includes first determining the proper implant thickness needed to match physiological valgus. The surgeon prepares the site arthroscopically, removing excess cartilage and removing the medial meniscus to the medial ring, using a portal of about 1 cm in order to provide suitable arthroscopic access while maintaining the presence of fluid in the joint. The implant can be initially molded ex vivo, using a mold selected from those available and including one or more embedded or attached fixation portions (e.g., anterior sutures or tabs), at which time it is inserted into the knee. The surgeon will then typically feel the implant once in position, to confirm that the implant is properly seated, and will extend the knee to provide varus stress on the lower leg, obtaining congruency as the implant continues to cure by finally molding both surfaces of the implant (to both the tibial surface and condyle, respectively).

In the preferred embodiment, the patient will have a diagnosis of osteoarthritis and have loss of cartilage on the articulating surface. A determination will be made of the amount of correction needed for the reestablishment of a normal angle of articulation. The ligaments will be balanced so that there is no loss of range of motion with the implant in place and the surface will be placed in such a position that the eventual resulting surface geometry reestablishes the same plane and orientation of the original articular surface.

Access to the site is preferably obtained in a minimally invasive way. In a particularly preferred embodiment, this is accomplished through arthroscopic means with arthroscopic portals. In an alternative embodiment, the access is accomplished by a mini arthrotomy with a small incision that allows access to the joint without sacrificing nerves, vessels, muscles or ligaments surrounding the joint. In the preferred embodiment fibrillated articulating cartilage that is degenerated is removed down to the subchondral surface.

A medial arthrotomy is created to provide access for the implant. This also provides an opening to use a smoothing device of the present invention on either the femoral and/or tibial surfaces. The smoothing device can be secured to the powered driver (e.g., a Triton brand reciprocating saw) by inserting the shaft of the device and tightening the collett on the driver. The speed of the driver is controlled in two ways, namely, by either limiting the air pressure delivered to the driver using an air regulator, and/or by a variable speed valve on the driver, which provides more speed (strokes per second) with increased depression of the control lever.

The smoothing device can be manipulated around and within the joint space, usually guided by placing an-index finger on the non-cutting side of the-blade. The blade is sufficiently flexible to permit it to be bended by finger pressure alone, without undue fatigue on the part of the surgeon. Ridges and shape points can be removed from the femur, while taking care not to cut through to trabecular bone. The relatively non-aggressive cutting surface of the device, relative to conventional rasps and rotating burrs, makes this easier to accomplish. Osteophytes should also be removed if they might impinge on the implant or limit range of motion.

Smoothness of the femoral and/or tibial surfaces can be judged in any suitable manner, including by finger palpation. When the surfaces are deemed smooth enough, the joint is thoroughly irrigated to remove any debris. Although typically powered, the excursion can be kept within a range sufficient to permit the surgeon's finger to be kept on the opposite (non-smoothing) surface of the blade-like device, in order to gently oscillate with it. This, combined with the desired flexibility of the device permit it to be moved around the joint, assuming different conformations, in order to smooth any particular surface.

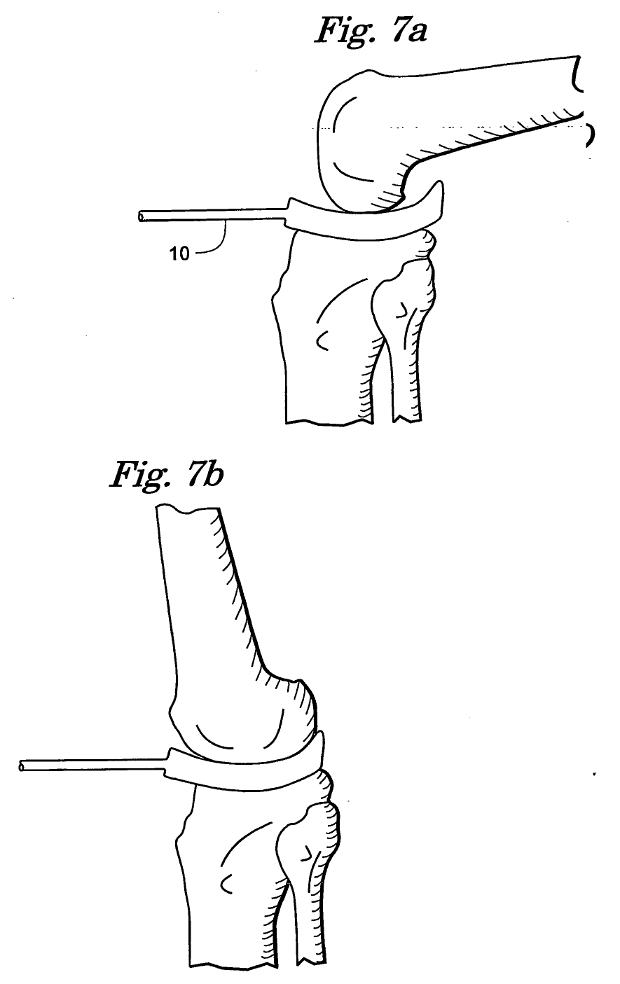

In an optional approach for smoothing the femoral surface and removing osteophytes, the smoothing device can be placed on the tibial plateau, in a manner that permits it to be contoured congruent with the femur. In this approach the device need not be powered by a reciprocator or oscillator. Instead, the joint can be put through a range of motion with gentle pressure on the medial compartment applied by placing varus pressure on the lower leg. This motion sweeps the femur across the textured surface of the device, thus smoothing the femoral surface. FIG. 7 provides a depiction of such alternative embodiment in which movement of the knee itself, ranging between flexion and extension, is used to achieve smoothing using a device as described herein. As shown, the smoothing device can be substantially thicker than a powered device (e.g., on the order of about 2 mm to about 4 mm, and is provided with a raised distal rim adapted to contact the posterior femoral surface, in order to remove osteophytes there. The distal rim serves the added benefit of helping to release the posterior capsule, which is particularly helpful for patients with extension deficits.

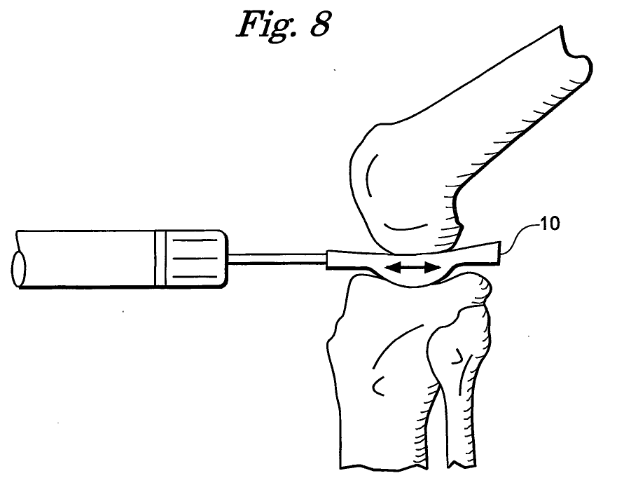

In yet another approach, as shown in FIG. 8, the smoothing device can be provided with a lower smoothing surface sized and adapted, upon its full range of excursion,-to smooth-and shape an area upon-that tibial-surface that closely resemble the shape and dimensions of the implant itself. The smoothing device can then be held in position upon the tibial plateau and reciprocated, e.g., using a suitable powered instrument.

In a related approach, the device and method of this invention can be used to achieve a predetermined, standardized conformation and/or degree of smoothness. For instance, suitably shaped devices and/or guides can be used to provide the condylar surface with a predetermined radius, corresponding to the particular implant to be used.

A system, including smoothing device, of this invention can be used to prepare the surface of the condyle prior to delivery of a polymeric implant as described herein. In use, the textured section of the smoother can be adjusted to the anatomy by bending so it can access areas not accessible with a straight rasp or shaver. For example, the bend allows the smoother to remove osteophytes from the posterior portion of the condyle, which would not be accessible with a commonly used rasp or shaver. The smoother can also be guided into contact with different areas of the bone by flexing and extending the joint. Since the operator need only guide the smoother into position and the motion of the smoother which causes the bone removal is provided by the reciprocating action of the aw, it can easily be used through 1 cm portal as well as a small arthrotomy. Since the abrasive surface is non-aggressive to soft tissue the surgeon can use a gloved indexed finger to direct, enhance and evaluate the smoothing of a bony surface.

The surface is dried and prepared for appropriate anchoring. This may include anchor points that give a mechanical lock or that alternatively simply supply horizontal and lateral stability. The surface may be prepared by drying and roughening in case a tissue adhesive is used. Pre-made anchors may be installed. These may be made of various metallic materials or of polymers and may consist of pegs that would extend up through the implant to anchor it to the underlying surface. This surrounding subchondral bone may be roughened to enhance tissue ingrowth or implant adhesion.

Once appropriate size, shape have been determined, an appropriate final implant can be selected and/or made, and in turn implanted and secured, e.g., in the manner described in Applicant's above-captioned co-pending published application.

1. A smoothing device for preparing one or more surfaces within an articulating joint site, the device comprising a substantially flat, straight or curved, blade having a proximal portion-adapted to be hand held and/or attached to a powered surgical instrument, and a distal portion having at least one major surface provided with a texture adapted to smooth cartilage within the joint site.

2. A device according to claim 1 wherein the device is adapted for use with one or more surfaces of the bones in the knee joint.

3. A device according to claim 2 wherein the device is adapted for use in smoothing the condylar surface.

4. A device according to claim 1 wherein the blade is fabricated from surgical stainless steel.

5. A device according to claim 4 wherein the distal portion is textured by providing either a plurality of closely spaced holes extending through the width of the blade or a plurality of pegs or ridges positioned upon the blade.

6. A device according to claim 5 wherein the device is adapted for use in a reciprocating saw instrument, and fabricated to retain a predetermined curved shape.

7. A device according to claim 6, wherein the device has an overall length of between about 100 mm and 150 mm, with a substantially distal portion having a length of between about 20 mm and about 40 mm.

8. A device according to claim 7 wherein the blade with is between about 5 mm and about 10 mm, and has a thickness of between about 0.3 mm and about 5 mm.

9. A device according to claim 8 wherein the proximal portion of the device is provided in the form of generally circular shaft, adapted to be fixably and releasably positioned within a powered surgical instrument.

10. A device according to claim 9 wherein the powered surgical instrument is adapted to operate the blade at an excursion distance of between about ½ mm and about 10 mm.

11. A method of smoothing bone, comprising providing a device according to claim 1 and using the device to smooth at least one surface of an articulating joint, prior to providing a surface implant material.

12. A method according to claim 11 wherein the device is adapted for use in smoothing the condylar surface of the knee.

13. A method according to claim 11 wherein the device is adapted for use in smoothing the tibial surface of the knee.

14. A method according to claim 11 wherein the blade is fabricated from surgical stainless steel.

15. A method according to claim 11 wherein the distal portion is textured by providing a either plurality of closely spaced holes extending through the width of the blade or a plurality of pegs or ridges positioned upon the blade.

16. A method according to claim 15 wherein the device is adapted for use in a reciprocating saw instrument, and fabricated to retain a predetermined curved shape.

17. A method according to claim 16, wherein the device has an overall length of between about 100 mm and 150 mm, with a substantially distal portion having a length of between about 20 mm and about 40 mm.

18. A method according to claim 17 wherein the blade with is between about 5 mm and about 10 mm, and has a thickness of between about 0.3 mm and about 5 mm.

19. A method according to claim 18 wherein the proximal portion of the device is provided in the form of generally circular shaft, adapted to be fixably and releasably positioned within a powered surgical instrument.

20. A kit comprising one or more devices according to claim 1, and further comprising one or more polymeric implants for fixation to the surface of opposing bone, in a manner that contacts the smoothed bone over the course of the implant's use.
